(19) United States
(12) Patent Application Publication
(10) Pub. No.: US 2016/0374616 A1
(43) Pub. Date: Dec. 29, 2016

(54) ELECTRODE CONTACT QUALITY

(71) Applicant: DAQRI, LLC, Los Angeles, CA (US)

(72) Inventors: Brian Mullins, Altadena, CA (US); Teresa Ann Nick, Woodland Hills, CA (US)

(21) Appl. No.: 14/749,384

(22) Filed: Jun. 24, 2015

(51) Int. Cl. A61B 5/00; G06T 19/00; G06T 19/20; A61B 5/0478

(52) U.S. Cl. CPC A61B 5/6843; A61B 5/0478; G06T 19/006; G06T 19/20

(57) ABSTRACT

A system and method for determining a quality of contact of an electroencephalogram (EEG) electrode is described. An electrode is connected to a user to detect brainwave activity from the user. An EEG application computes a voltage of the electrode, computes a derivative of the voltage of the electrode, computes a coefficient of variation from the derivative of the voltage, and determines a quality of contact of the electrode to the user based on the coefficient of variation.

### TECHNICAL FIELD

The subject matter disclosed herein generally relates to the processing of data. Specifically, the present disclosure addresses systems and methods for identifying contact quality of electrodes.

### BACKGROUND

Electroencephalogram (EEG) is a method to record electrical activity of the brain of a person. The voltage fluctuations are measured and recorded by applying electrodes to the scalp or forehead of a person. Wet electrodes are conventionally applied to the skin. However, wet electrodes require applying a conductive gel between the electrode and the skin of the person. Applying the conductive gel requires a lot of time and can be cumbersome to clean up.

Dry electrodes are more convenient than wet electrodes because they can be applied to the skin of the person without requiring conductive gel. However, the dry electrodes temporarily lose contact with the skin when the person moves during an EEG recording session. Therefore, data recorded from dry electrodes may not be as reliable as data recorded from wet electrodes.

### BRIEF DESCRIPTION OF THE DRAWINGS

Some embodiments are illustrated by way of example and not limitation in the figures of the accompanying drawings.

FIG. 1 is a block diagram illustrating a device suitable for determining electrode contact quality and generating augmented reality content based on the electrode contact quality, according to some example embodiments.

FIG. 2 is a block diagram illustrating modules (e.g., components) of the EEG application in the device of FIG. 1, according to some example embodiments.

FIG. 3 is a block diagram illustrating modules (e.g., components) of an electrode contact quality module of the EEG application of FIG. 2, according to some example embodiments.

FIG. 4 is a block diagram illustrating modules (e.g., components) of the augmented reality application of FIG. 2, according to some example embodiments.

FIG. 5A is is a diagram illustrating of a side view of a head of a user with dry electrodes applied to the forehead of the user.

FIG. 5B is is another diagram illustrating of a side view of a head of a user with dry electrodes applied to the forehead of the user with poor contact.

FIG. 6A is a diagram illustrating of a top view of a head of a user with electrodes applied to the head of the user.

FIG. 6B is a diagram illustrating an example of virtual content displayed in the device of FIG. 1 based on a physical identifier, according to some example embodiments.

FIG. 6C is a diagram illustrating an example of changes to virtual content in the device of FIG. 1 based on electrode contact quality, according to some example embodiments.

FIG. 7 is a flowchart of a method, in accordance with an example embodiment, of generating a visualization corresponding to a physical object and a mental state of a user.

FIG. 8 is a flowchart of a method, in accordance with an example embodiment, of identifying electrode contact quality.

FIG. 9 is a flowchart of a method, in accordance with another example embodiment, of identifying electrode contact quality.

FIG. 10 is a flowchart of a method, in accordance with an example embodiment, of changing virtual content based on a change in the electrode contact quality.

FIG. 11 is a block diagram illustrating components of a machine, according to some example embodiments, able to read instructions from a machine-readable medium and perform any one or more of the methodologies discussed herein.

### DETAILED DESCRIPTION

Example methods and systems are directed to identifying contact quality of EEG electrodes and changing virtual objects based on the contact quality. Examples merely typify possible variations. Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will be evident to one skilled in the art, however, that the present subject matter may be practiced without these specific details.

Augmented reality applications allow a user to experience information, such as in the form of a three-dimensional virtual object displayed in a transparent lens or in a non-transparent display overlaid on a picture of a physical object captured by a camera of a device. The physical object may include a visual reference that the augmented reality application can identify. The three-dimensional virtual object may be selected based on the recognized visual reference. A rendering of the visualization of the three-dimensional virtual object may be based on a position of the display relative to the visual reference. The virtual object may change based on a mental state of a user and the contact quality of the electrodes. The mental focus state of a user may be determined by recording different types of brain waves from dry electrodes connected to a forehead of the user and performing a computation on the recorded data. For example, if the mental focus state of the user identifies the user as focused but the contact quality deteriorates, the device may display a first virtual object based on the “good” contact quality and alert the user of the “bad” contact quality. The alert may be displayed in the form of a visual notification in a display. In another example, if the mental focus state of the user identifies the user as not-focused with a “good” contact quality, the device may display a second virtual object different from the first virtual object.

The present application describes a method for qualifying electrode contact quality due to poor contact with the skin of the user. The contact quality algorithm reports when electrode signals degrade due to removal of the electrodes (in a headband or a helmet) from the head or poor signal due to other events, such as a movement of the headband. For example, the algorithm returns PoorQuality=TRUE if the signal is poor quality and FALSE otherwise.

The voltage recorded by an open circuit typically drifts over large amplitudes at low frequencies. This can be quantified as a large coefficient of variation (CV) of the derivative of the voltage. A threshold can be set at an experimentally determined maximum CV and return TRUE (poor quality signal) if the CV is higher than this threshold.

Front-end digital signal processors compensate open-circuit large amplitude fluctuations such that the voltage eventually flatlines. This can be quantified as a very small coefficient of variation of the derivative of the voltage. A threshold can be set at an experimentally determined minimum CV and return TRUE (poor quality) if the CV is lower than this threshold.

As the front-end digital signal processor compensates the signal, the CV can cross into “good quality” range. This is dealt with by requiring that the signal be good for a specific duration of time (converted to cycles from time and applied as the minimumFalseCount) for the algorithm to indicate that the signal is good (return FALSE).

The present application describes the use of the coefficient of variation of the derivative as part of a contact quality algorithm and the use of maximum CV to reject open-circuit signal irrespective of the use any front-end digital signal processor.

A system and method for qualifying electrode contact quality is described. In an example embodiment, a device includes at least one electrode and a hardware processor. The electrode is connected to the skin of a user to detect brainwave activity from the user. The hardware processor comprises an electroencephalogram (EEG) application configured to compute a voltage of the electrode, to compute a derivative of the voltage of the electrode, to compute a coefficient of variation from the derivative of the voltage, and to determine a quality of contact of the electrode to the user based on the coefficient of variation.

In one example embodiment, the hardware processor determines a maximum coefficient of variation and a minimum coefficient of variation for the electrode. The hardware processor determines that the coefficient of variation is between the minimum coefficient of variation and the maximum coefficient of variation. The hardware processor then identifies the voltage as valid and a contact of the electrode as good quality in response to determining that the coefficient of variation is between the minimum coefficient of variation and the maximum coefficient of variation.

In one example embodiment, the hardware processor determines that the coefficient of variation is between the minimum coefficient of variation and the maximum coefficient of variation. The hardware processor identifies the voltage as not valid and a contact of the electrode as bad quality in response to determining that the coefficient of variation outside a range between the minimum coefficient of variation and the maximum coefficient of variation.

In one example embodiment, the hardware processor computes the standard deviation of the derivative of the voltage of the electrode, computes the mean of the derivative of the voltage of the electrode, and computes the coefficient of variation of the electrode by dividing the standard deviation of the derivative of the voltage of the electrode by the mean of the derivative of the voltage of the electrode.

In one example embodiment, the quality of contact includes a Boolean value. For example, the Boolean value includes a good quality and a bad quality. The hardware processor determines a minimum false time duration as a time for a good contact quality of the electrode before identifying a bad contact quality of the electrode. The hardware processor identifies the bad contact quality of the electrode in response to identifying bad contact quality for a period of time exceeding a predefined number of voltage computation cycles.

In one example embodiment, the device includes a camera that captures a reference identifier from a physical object. The hardware processor includes an augmented reality application to identify a virtual object associated with the reference identifier, to display the virtual object in a display of the device, in response to a relative movement between the device and the physical object caused by a user, to modify the virtual object based on the quality of contact of the electrode to the user.

In one example embodiment, the EEG application identifies a change in a state of mind of the user of the device. The augmented reality application modifies the virtual object based on the change in the state of mind of the user of the device.

In another example embodiment, a non-transitory machine-readable storage device may store a set of instructions that, when executed by at least one processor, causes the at least one processor to perform the method operations discussed within the present disclosure.

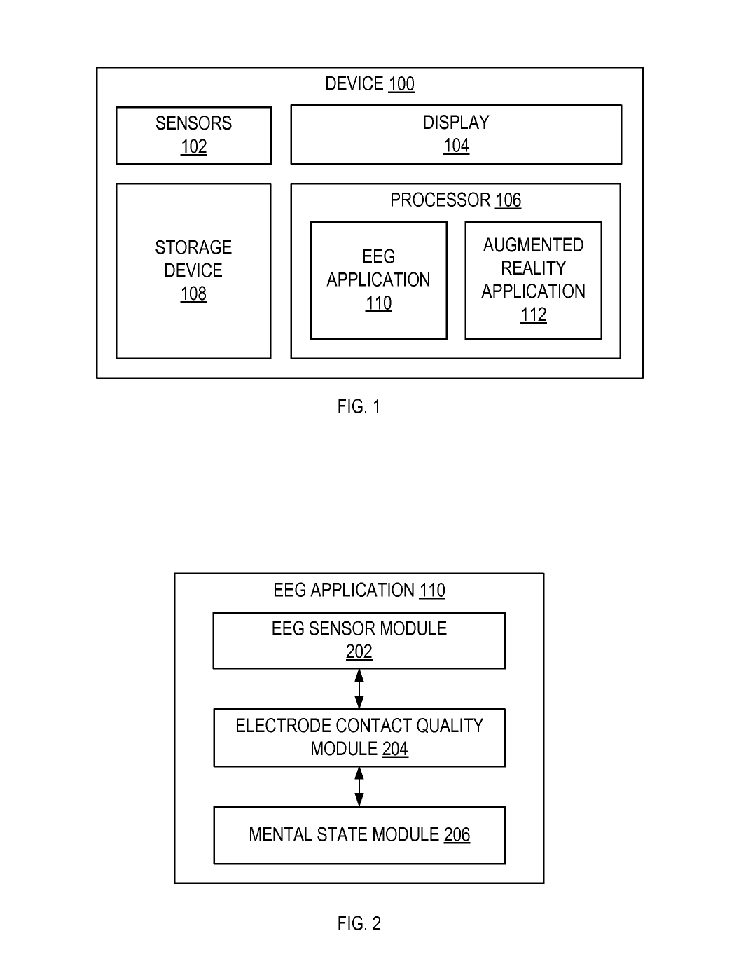

FIG. 1 is a block diagram illustrating a device suitable for electrode contact quality and a mental state of the user and generating augmented reality content based on the mental state and electrode contact quality, according to some example embodiments. The device 100 may include sensors 102, a display 104, a processor 106, and a storage device 108. For example, the device 100 may be a desktop computer, a vehicle computer, a tablet computer, a navigational device, a portable media device, a smart phone of a user, or a user wearable computing device (e.g., glasses), or a head mounted device (HMD). The user may be a human user (e.g., a human being), a machine user (e.g., a computer configured by a software program to interact with the device 100), or any suitable combination thereof (e.g., a human assisted by a machine or a machine supervised by a human).

The sensors 102 may include at least two electrodes that measure electrical activity from a human. For example, the sensors 102 may include a first and a second electrode connected to a forehead of a user. The sensors 102 measure EEG (electroencephalography) waves of brains, EMG (electromyography) waves of muscles, and EOG (electro-oculogram) waves of eyes. The sensors 102 can also be used to monitor brainwaves through EEG by detecting electrical signals about the user's present level of focus or focus state. The sensors may be implemented, for example, by using a headset or a helmet worn by the user. In another example, the sensors 102 can be used to monitor facial muscles to detect facial expressions of the user.

In another example embodiment, the sensors 102 may also include: an optical sensor (e.g., a charged-coupled device (CCD)), an orientation sensor (e.g., gyroscope), and/or an audio sensor (e.g., a microphone). For example, the device 100 may include a front-facing camera for tracking eyes movement and facial expression of the user, and a rear-facing camera for capturing a picture or a video of a physical object (or another displayed virtual object). It is noted that the sensors 102 described herein are for illustration purposes and the sensors 102 are thus not limited to the one described. In another example, sensors 102 may not be physically connected to the device 100 but are instead coupled to the device 100 via wireless means such as Wi-Fi and Bluetooth O.

The display 104 may include, for example, a touchscreen display configured to receive a user input via a contact on the touchscreen display. In another example, the display 104 may include a screen or monitor configured to display images generated by the processor 106. In another embodiment, the display 104 may be transparent so that the user can see through the display 104.

The processor 106 may include an EEG application 110 and an augmented reality (AR) application 112. The EEG application 110 may determine a mental state of the user such as a level of focus of the user (e.g., focused, distracted) based on outputs from the sensors 102. The level of focus may be based on the intensity or pattern of the outputs of the sensors 102. The level of focus may be classified in a spectrum from intensively focused to very distracted and non-focused. The level of focus may be predefined relative to the range of outputs from the sensors 102. The EEG application 110 further determines contact quality of the sensors 102 (e.g., dry electrodes). For example, the EEG application 110 uses a contact quality algorithm (described further below) to identify the quality of the signal from the electrodes to determine whether to include the signals in the computation to determine the mental state of the user.

The AR application 112 generates a display of a virtual object (three-dimensional or two-dimensional model) in the display 104 of the device 100. In another example, the AR application 112 generates a display of the virtual object overlaid on an image of a physical object captured by a camera (not shown) of the device 100. The virtual object may be selected or generated based on the level of focus of the user. The virtual object and features of the virtual object may be further manipulated based on a change in the level of focus of the user. In another example embodiment, the virtual object may be further manipulated (e.g., by the user) by moving the physical object relative to the camera lens of the device 100. Similarly, the display of the virtual object may be manipulated (e.g., by the user) by moving the camera lens of the device 100 relative to the physical object.

In one example embodiment, the EEG application 110 identifies the intensity or pattern of the different types of electric waves discharged the brain of the user over a short period of time (a sampling period). At least two electrodes may be placed on the forehead of the user. Each electrode may be configured to measure different types of waves. For example, Delta waves are most present during sleep. Theta waves are associated with sleep, deep relaxation, and visualization. Alpha waves occur when relaxed and calm. Beta waves occur when actively thinking or problem-solving. Gamma waves occur when involved in higher mental activity and consolidation of information. The mental focus state application 110 identifies a level of focus of the user based on the outputs of the sensors 102. For example, the EEG application 110 may use EEG electrodes alone or in combination with other sensing devices (microphone, camera, and heart rate monitor).

In one embodiment, the augmented reality application 112 may identify a visual reference on the physical object and tracks the location of the visual reference within the display 104 of the device 100. The visual reference may also be referred to as a marker and may consist of an identifiable image, symbol, letter, number, machine-readable code. For example, the visual reference may include a bar code, a quick response (QR) code, or an image that has been previously associated with the virtual object.

The augmented reality application 112 may generate and display a virtual object overlaid on top of an image or picture of the physical object in the display 104. The virtual object may be generated based on the visual reference and the level of focus of the user. Each virtual object may correspond to a unique visual reference and corresponding level of focus (e.g., unique to that virtual object within the augmented reality application 112). In another embodiment, the augmented reality application 112 renders the the virtual object based a position and an orientation of the device 100 relative to the visual reference of the physical object.

The storage device 108 may be configured to store a database of visual references, virtual objects corresponding to the visual references, features of the virtual objects corresponding to the virtual objects, and corresponding focus levels. The features of the virtual objects can change with the level of focus of the user. For example, the color of the virtual chair can change from blue to red as the user becomes more focused. The virtual chair may be displayed in a blue color if the user is relaxed. In another example, features of the virtual object change when the features are present in a specific area (also referred to as focus area) in the display 104. For example, the visual reference may include a machine-readable code or a previously identified image (e.g., a picture of shoe). The previously identified image of the shoe may correspond to a three-dimensional virtual shoe that can be viewed from different angles by manipulating the position of the device 100 relative to the picture of the shoe. Features of the three-dimensional virtual shoe may include selectable icons on the three-dimensional virtual shoe. An icon may be selected or activated by moving (e.g., repositioning, reorienting, or both) the device 100 to display the icon within a focus area of the display 104. For example, the focus area may be a central area of the display 104, a corner of the display 104, an edge of the display 104, or any suitable combination thereof.

In one embodiment, the device 100 may communicate over a network (not shown) with a server (not shown) to retrieve a portion of the database of visual references, corresponding three-dimensional virtual objects, corresponding features of the three-dimensional virtual objects, and corresponding level of focus. The network may be any network that enables communication between or among machines, databases, and devices (e.g., the device 100). Accordingly, the network may be a wired network, a wireless network (e.g., a mobile or cellular network), or any suitable combination thereof. The network may include one or more portions that constitute a private network, a public network (e.g., the Internet), or any suitable combination thereof.

Any one or more of the modules described herein may be implemented using hardware (e.g., a processor of a machine) or a combination of hardware and software. For example, any module described herein may configure a processor to perform the operations described herein for that module. Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices.

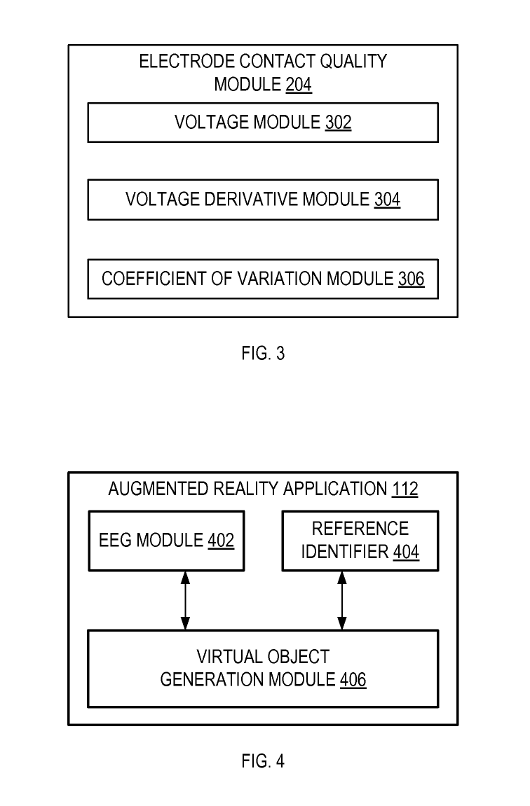

FIG. 2 is a block diagram illustrating modules (e.g., components) of the EEG application in the device of FIG. 1, according to some example embodiments. The EEG application 110 includes an EEG sensor module 202, an electrode contact quality module 204, and a mental state module 206.

The EEG sensor module 202 captures outputs from the sensors 102. The EEG sensor module 202 may capture electric waves generated by a brain of the user by using EEG electrodes in contact with the forehead of the user. As previously described, the sensor module 202 may also capture outputs from other types of sensors such as a heart rate monitor or a facial muscle monitor to further supplement outputs from the EEG electrodes. In another embodiment, the sensors 102 may include a camera to detect the gaze of the user and determine where the user is look on the display 104.

The EEG sensor module 202 captures data from the sensors 102 (dry electrodes) during a sampling period (e.g., 4 ms to 1000 ms). The sample rate may be, for example, 250 Hz. Thus, the sampling module 204 captures a sample of outputs from the sensors at the sample rate during a sampling period. The sample outputs may then be used as a baseline or a reference for the user.

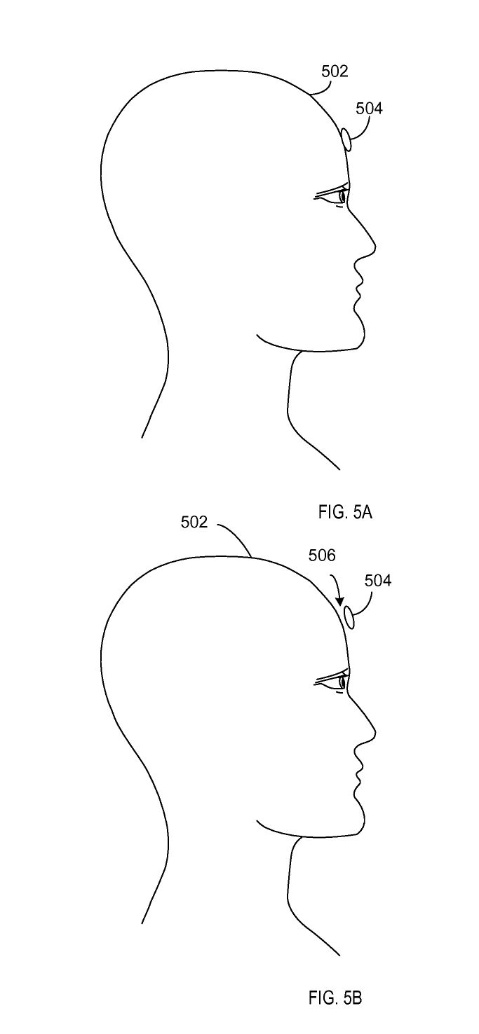

The electrode contact quality module 204 determines, qualifies, and identifies the electrode contact quality. For example, the electrode contact quality module 204 uses an electrode contact quality algorithm to determine the contact quality of the electrode and to ensure that only data associated with “good” contact quality is recorded and used in the computation of the mental state module 206. An example of the electrode contact quality algorithm is illustrated further below with respect to FIGS. 3, 8 and 9.

The mental state module 206 receives an identification or signal quality qualification from the electrode contact quality module 204 and also data from other sensors. For example, the mental state module 206 may measure the intensity of electrical signals of Alpha waves and the quality of the signals in combination with the heart rate of a user to determine the user's relaxed state of mind. In another example embodiment, the intensity may be based on a statistical computation (e.g., average or median) of one or more outputs from selected sensors.

The mental state module 206 may thus determine and identify, for example, a level of focus of the user based on brain activity data and whether the data is “good” reliable data as determined by electrode contact quality module 204. For example, the mental state module 206 may determine that the user is very focused up to a certain point in time when the signal quality degraded. In another example embodiment, the mental state module 206 may determine a change in the level of focus of the user based on changes in the signal quality from the electrodes. For example, the mental state module 206 may determine that the user who was previously focused has now become distracted.

FIG. 3 is a block diagram illustrating modules (e.g., components) of an electrode contact quality module of the EEG application of FIG. 2, according to some example embodiments. The electrode contact quality module 204 includes a voltage module 302, a voltage derivative module 304, and a coefficient of variation module 306. The voltage module 302 retrieves the voltage from the electrode. For example, a front-end analog-to-digital conversion module may compute the differential voltage between two electrodes. The voltage derivative module 304 computes a derivative of the voltage obtained by the voltage module 302. For example, a firmware or mobile application process may compute the differences between subsequent voltage samples. The coefficient of variation module 306 calculates a coefficient of variation based on the derivative of the voltage. For example, a firmware or mobile application process may compute the ratio of the standard deviation of the voltage differences divided by the mean of the voltage differences.

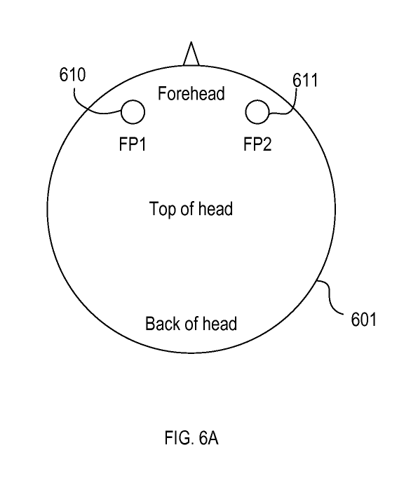

FIG. 4 is a block diagram illustrating modules (e.g., components) of the augmented reality application of FIG. 2, according to some example embodiments. The augmented reality application 112 includes an EEG module 402, a reference identifier module 404, and a virtual object generation module 406.

The EEG module 402 communicates with the EEG application 110 to access electrode contact quality as determined by the electrode contact quality module 204 and a present mental state of the user as determined by the mental state module 206. For example, the electrode contact quality module 204 may indicate that the quality of the electrode is good. The mental state module 206 may indicate that the user is very focused. The mental state module 206 may further identify changes in the mental state or level of focus of the user.

The reference identifier module 404 identifies a visual reference on a physical object captured by sensors 102 of the device 100. For example, a camera of the device 100 captures an image of a physical object, such as a page on a newspaper. The page on the newspaper may include an article and a picture. The picture may have been already identified as a visual reference in the storage device 108. The picture may be associated with a corresponding three-dimensional model of an object (e.g., a virtual sofa).

The virtual object generation module 406 generates and displays a visualization of a three-dimensional virtual object engaged with an image of the physical object captured by the sensor 102 of the device 100 (e.g., the virtual sofa floats and rotates on top of the magazine page). The virtual object may be based on the visual reference (e.g., a furniture ad in the magazine page). In one embodiment, each virtual object may be uniquely associated with a visual reference. The virtual object generation module 304 renders the visualization of the virtual object based a position of the device 100 relative to the visual reference. In another embodiment, attributes of the virtual object may be based on the level of focus of the user. For example, the virtual object generation module 406 may generate a blue color sofa when the level of focus of the user indicates that the user is distracted. Similarly, the virtual object generation module 406 may generate a red color sofa when the level of focus of the user indicates that the user is very focused.

In yet another embodiment, the virtual object generation module 406 generates a virtual object based on a change in the level of focus of the user and a “good” electrode contact quality. For example, a blue color car may morph into a red color sofa when the state of mind of the user indicates that the user is getting distracted.

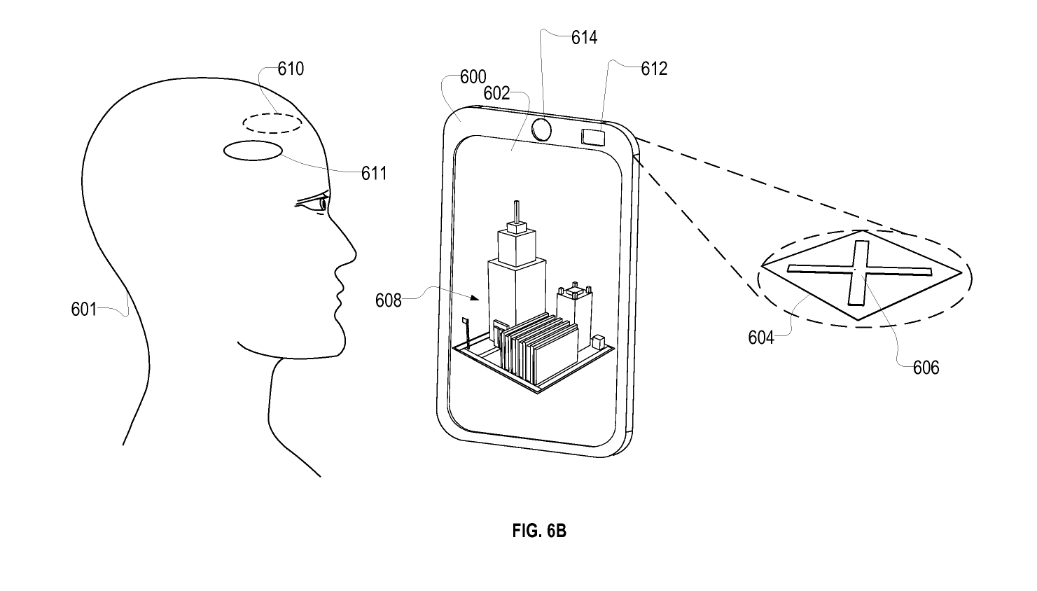

FIG. 5A is is a diagram illustrating of a side view of a head of a user with dry electrodes applied to the forehead of the user. A dry electrode 504 is in contact with the forehead of the user 502. The electrode contact quality module 204 may identify the contact as a “good” quality contact based on the data recorded from the dry electrode 504.

FIG. 5B is is another diagram illustrating of a side view of a head of a user with dry electrodes applied to the forehead of the user. The dry electrode 504 is not in contact with the forehead of the user 502. A gap 506 is shown between the dry electrode 504 and the forehead of the user 502. The electrode contact quality module 204 may identify the contact as a “bad” quality contact based on the data recorded from the dry electrode 504.

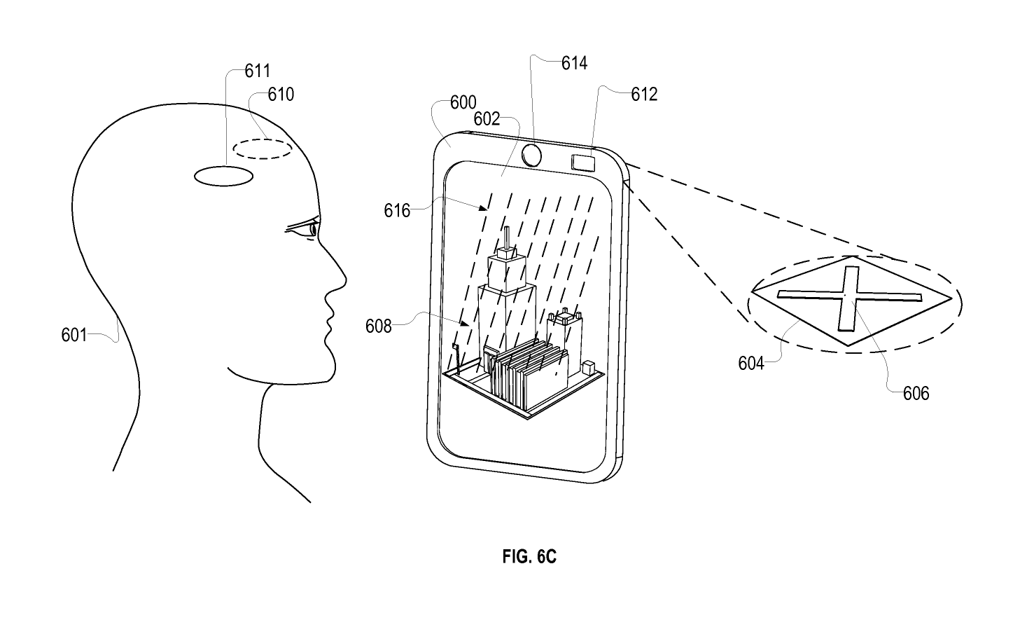

FIG. 6A is a diagram illustrating of a top view of a head of a user 601 with electrodes 610, 611 applied to the head of the user 601. The electrode contact quality algorithm may be applied to data from both electrodes individually or both simultaneously. The electrode contact quality algorithm may use data from both electrodes initially and may also dynamically switch between the electrode 610 and 611 depending of the quality of the data from the respective electrodes.

FIG. 6B is a diagram illustrating an example of virtual content displayed in the device of FIG. 1 based on a physical identifier, according to some example embodiments. The user 601 is equipped with electrodes 610 and 611 connected to the forehead of the user 601. As previously described, electrodes 610 and 611 may include other types of measuring devices for measuring facial muscle activity and heart rate activity among others. Electrodes 610 and 611 may be physically coupled via wires to a device 600 (e.g., mobile communication device). In another example, electrodes 610 and 611 may communicate with the device 600 wirelessly using wireless communication means (e.g., Bluetooth ®, ZigBee ®).

The user 601 points a rear camera 612 of the device 600 towards a physical object 604 having a visual reference 606. As previously described, the visual reference 606 may include a picture, a machine-readable code, or any other identifier unique to the augmented reality application 112 in the device 600. The physical object 604 may be, for example, a page of a magazine or newspaper. In another embodiment, the physical object 604 and the visual reference 606 may be combined together (e.g., a poster or a cup). In such case, the three-dimensional physical object may be used as a visual reference. For example, a three-dimensional object such as a cup having a specific pattern or design may be used as a visual reference. The device 600 captures an image or a picture of the physical object 604 and the visual reference 606 using the rear camera 612.

The device 600 generates a visualization of a three-dimensional virtual object in a display 602 of the device 600 based on outputs from sensors 610 and the visual reference 606. For example, the device 600 may determine that the user 601 is geographically located at an architectural firm. The device 600 determines from the sensors 610 that the level of focus of the user 601 corresponds to a focused state (as opposed to a distracted state) and that the quality of the electrode is “good.” In another embodiment, a front facing camera 614 of the device 600 may further enhance and provide additional data on the state of mind of the user 601. For example, the device 600 may obtain a live picture of the user 601 using the front facing camera 614 to determine a smile or a frown. In another example, the front facing camera 614 may be used for facial recognition to determine the identity of the user 601. The device 600 may retrieve preferences from the user 601 such as, for example, favorite colors or items. In another example, the device 600 determines, identifies, and manipulates a virtual object to be displayed in the display 602 based on a combination of the geographic location of the device 600 (e.g., office, home, restaurant, city, country), time of capture (e.g., morning, afternoon, evening, holiday, weekend) of the visual reference 606, orientation (e.g., portrait or landscape, how close) of the device 600 relative to the visual reference 606, identification of the user 601 (e.g. using facial recognition, or login information), preferences of the user 601 (e.g., favorite color, favorite type of music) social network information (e.g., number of friends, interests, proximity of friends, postings) related to the user 601, outputs from sensors 610 (e.g., EEG brain waves, EMG muscles waves, EOG eyes waves, heart rate, blood pressure), and the visual reference 606.

The device 600 may then generate a visualization of a three-dimensional virtual object engaged with a picture of the physical object 604 based on the level of focus the user 601. In the present example, a three-dimensional model of a building 608 is rendered on top of an image of the physical object 604 in the display 602 for a focused state. In another example, if the user's state of mind is relaxed, the device 600 may generate a three-dimensional model of a vacation home rendered on top of an image of the physical object 604 in the display 602. As such, the device 600 determines a virtual object to be displayed in the display 602 in response to the captured image of the visual reference 606 and the present level of focus of the user 601.

FIG. 6C is a diagram illustrating an example of changes to virtual content in the device of FIG. 1 based on electrode contact quality, according to some example embodiments. The device 600 determines a change in the state of mind of the user 601 (e.g., from focused to distracted). The device 600 then generates a change in the visualization of the three-dimensional virtual object in the display 602 of the device 600 based on the change in the level of focus of the user 601 in response to changes in outputs from sensors 610 and the front facing camera 614. For example, virtual rain 616 over the building 608 may be dynamically animated in the display 602 when the device 600 detects that the user 601 has frowned and is unfocused.

As such, changes of the already displayed three-dimensional virtual object in the display 602 are determined based on the changes in the state of mind of the user 601. In another example, the color of the building 608 may change to a lighter hue when the user 601 becomes more relaxed while looking at the building 608. In another example, the texture of the building 608 may change to a rougher texture when the user 601 becomes agitated.

In another embodiment, the device 600 may include a transparent display (not shown) that may be used to identify a physical object or a particular location on the physical object. In one example, the transparent display may be mounted to a head of the user (e.g., via eyeglass mount or headgear mount). In another example, the transparent display may be a handheld device that the user 601 holds and looks through to see a physical object behind the transparent display. The rear facing camera of the device 600 may recognize physical objects being looked by the user (e.g., by comparing an image of the physical object with a reference image). In particular, the position and orientation of the transparent display with respect to the user and the physical object may be used to determine a line of sight of the user. Using the determined line of the sight of the user, the device can identify in real time the physical objects being looked and in particular which part of the physical object is being looked.

Once the device 600 identifies that the recognized physical object or the part of the recognized physical object corresponds to a pre-identified physical object or pre-identified part of the physical object, the device may trigger a corresponding action (e.g., sending an email, generating a sound, etc.) based on the state of mind of the user 601. For example, the device 600 detects the user 601 looking through the transparent display to a bottom portion of a television set. The device 600 recognizes the television set and determines that the bottom portion of the television set (being looked at by the user 601) is associated with an action corresponding to generating a communication to the television set to switch the TV on or off If the user 601 has looked at the bottom portion of the television set for at least several seconds and the state of mind indicates that the user is focused, the device 600 generates a corresponding signal to turn on or off the television set.

In another example, the device 600 may display a virtual menu of TV channels overlaid on the TV based on the state of mind of the user 601. For example, if the user is excited, the menu of TV channels may include sports channels and action movies. In another example, the user 601 may look through a transparent display of the device 600 to a radio device. Similarly, a virtual menu of music channels may be displayed over the radio device based on the state of mind of the user 601. For example, the device 600 may display a virtual menu of classical or relaxing music channels when sensors 610 indicate that the user 601 is relaxed or sleepy.

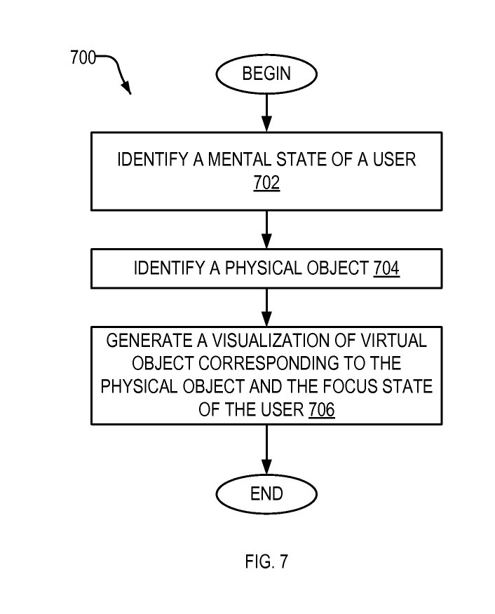

FIG. 7 is a flowchart of a method, in accordance with an example embodiment, of generating a visualization corresponding to a physical object and a mental state of a user. In operation 702, a present mental state (e.g., focused, distracted, worried, sleepy, relaxed, excited) of the user is identified based on the intensity of outputs of sensors connected to the forehead of the user. The output may include, for example, electric brain waves. In one embodiment, the operation 702 may be performed using the EEG application 110 of the device 100 of FIG. 1.

In operation 704, an image of a physical object captured the device 100 is recognized or identified. In one embodiment, the operation 704 may be performed by the reference identifier module 404 that identifies a visual reference on the physical object.

In operation 706, the augmented reality application 112 generates and displays a virtual object engaged (e.g., overlaid on top of) with an image of the physical object. The virtual object corresponds to the visual reference and the level of focus state (or mental state) of the user and the quality of the electrode contacts. In one embodiment, the virtual object generation module 406 renders the virtual object based a position of the display relative to the visual reference.

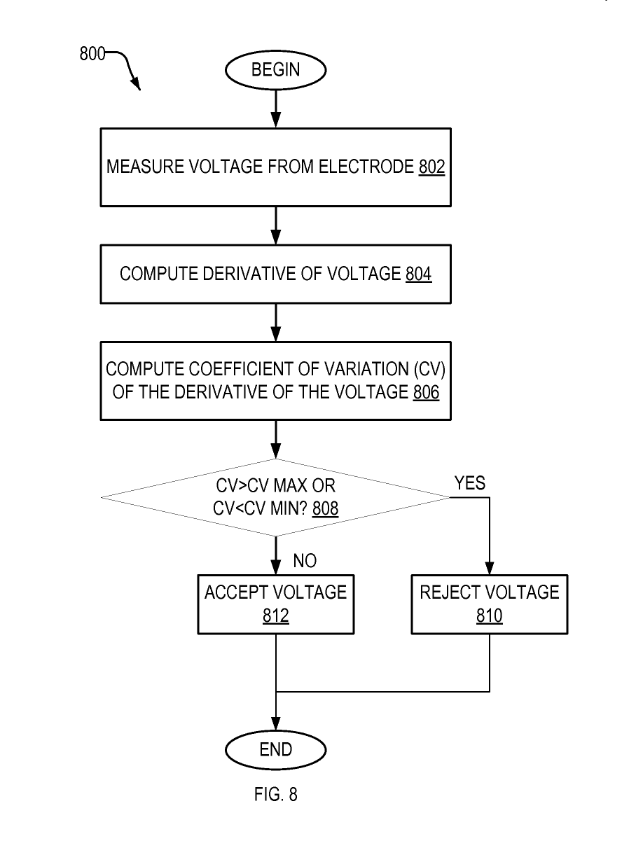

FIG. 8 is a flowchart of a method, in accordance with an example embodiment, of identifying electrode contact quality. At operation 802, the voltage from the electrode(s) is measured. At operation 804, the derivative of the voltage is computed. At operation 806, the coefficient of variation (CV) of the derivative of the voltage is calculated. At operation 808, the calculated coefficient of variation is compared with a predetermined maximum coefficient of variation. The maximum CV may have been previously empirically determined based on previous data (e.g., historical data). If the current CV exceeds the maximum CV or the current CV is below the minimum CV, the voltage from the electrode is rejected. If the current CV is below the maximum CV or the current CV exceeds the minimum CV, the voltage from the electrode is accepted into the mental state module 206.

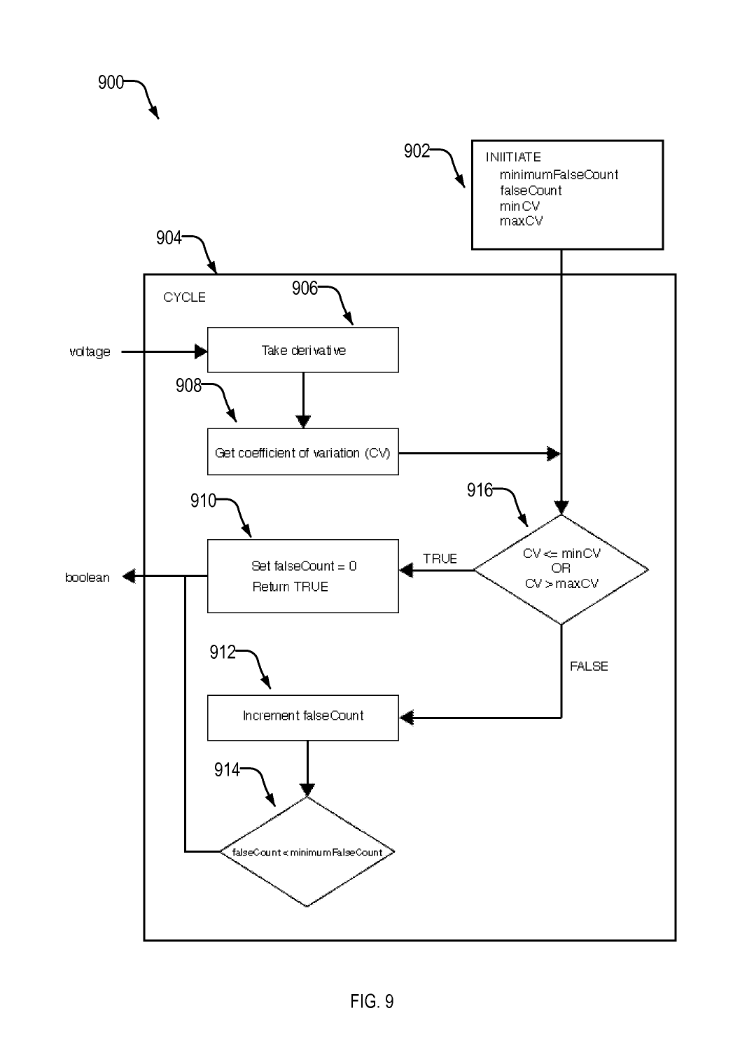

FIG. 9 is a flowchart of a method, in accordance with another example embodiment, of identifying electrode contact quality. At operation 902, the following variables are initialized minimumFalseCount, falseCount, min CV, max CV. minimumFalseCount represents minimumFalseDuration/refreshInterval. minimumFalseDuration represents the time for the contact quality to be good before it actually returns that poor quality is FALSE. The falseCount represents the number of cycles that the contact quality is determined to be good. The minCV and maxCV are experimentally determined based on hardware (e.g., signal processors) used to process the signal from the electrode.

A cycle 904 includes operations 906, 908, 910, 912, 914, and 916. At operation 906, the derivative of the voltage is computed. At operation 908, the coefficient of variation (CV) is calculated based on the derivative of the voltage. At operation 916, the contact quality algorithm determines whether the CV is less than minCV or greater than maxCV. In that case (if true), the contact quality algorithm sets the falseCount to 0 and returns TRUE. If the contact quality algorithm determines that CV is greater than minCV or less than maxCV, than the falseCount is incremented. At operation 914, if the falseCount is greater than the minimumFalse Count, the contact quality algorithm indicates good quality (poorQuality=FALSE). For example, if an electrode has poor contact, the measured voltage will have large variations that intermittently saturate the dynamic range of recording hardware, resulting in a large CV of the derivative of voltage. Some front-end devices may compensate and pull the voltage down toward zero, in which case the CV becomes very small over time. In the compensation case, there is a period during which the CV is in the acceptable range (between minCV and maxCV), even though the contact quality is poor. To address this issue, a signal is required to have an acceptable CV for a finite amount of time (defined by minimumFalseDuration) before it is actually labeled as good by the algorithm (poorQuality=FALSE).

The following is an example of a pseudo code for the contact quality algorithm described in the present application:

```
          Contact Quality Algorithm -
          initiated by main program once
          called on every graphics refresh cycle by main program
          begin initiation
              minimumFalseDuration = d //The time for contact quality
to be good before actually return that poor quality is FALSE
              refreshInterval = r
              minimumFalseCount = floor(d/r)
              falseCount = 0
              minCV = cv1 //Experimentally determined for a
particular hardware
              maxCV = cv2 //Experimentally determined for a
particular hardware to end initiation
          input: voltage
          output: boolean isPoorQuality
          begin cycle
              diff <- derivative(voltage)
              coefficientOfVariation <- StandardDeviation(diff)/
              Mean(diff)
              if (coefficientOfVariation > maxCV OR
coefficientOfVariation <= minCV){
                falseCount <- 0
                return TRUE
              }
              else {
                falseCount <- falseCount + 1
                return (falseCount < minimumFalseCount)
                //Indicate that contact quality is still poor during brief
apparently good quality periods
          end cycle
```

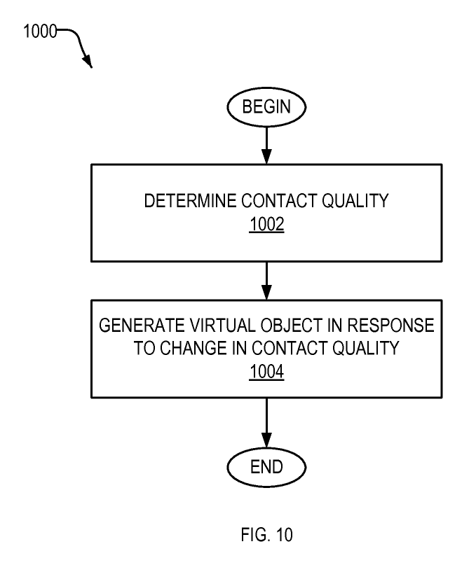

FIG. 10 is a flowchart illustrating an example operation of changing virtual content based on a change in the electrode contact quality, according to some example embodiments. In operation 1002, a change in the electrode contact quality is identified based on a change in the contact quality as determined by the electrode contact quality module 204 of FIG. 2.

In operation 1004, the virtual object generation module 506 changes a visualization of the previously displayed virtual object in the display based on the change in the electrode contact quality. In other words, an action may be performed on the virtual object in the display based on the change in the change in the electrode contact quality. The virtual object may temporarily disappear in response to a bad electrode contact quality. In other examples, the color of the virtual object may change from red to blue as the contact quality degrades. The virtual object may spin faster as the contact quality improves. In one embodiment, the virtual object generation module 406 renders the visualization of virtual object based a change in a position of the display relative to the visual reference.

### Modules, Components and Logic

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client, or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network and via one or more appropriate interfaces (e.g., APIs).

### Electronic Apparatus and System

Example embodiments may be implemented in digital electronic circuitry, or in computer hardware, firmware, software, or in combinations of them. Example embodiments may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

A computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

### Example Machine Architecture and Machine-Readable Medium

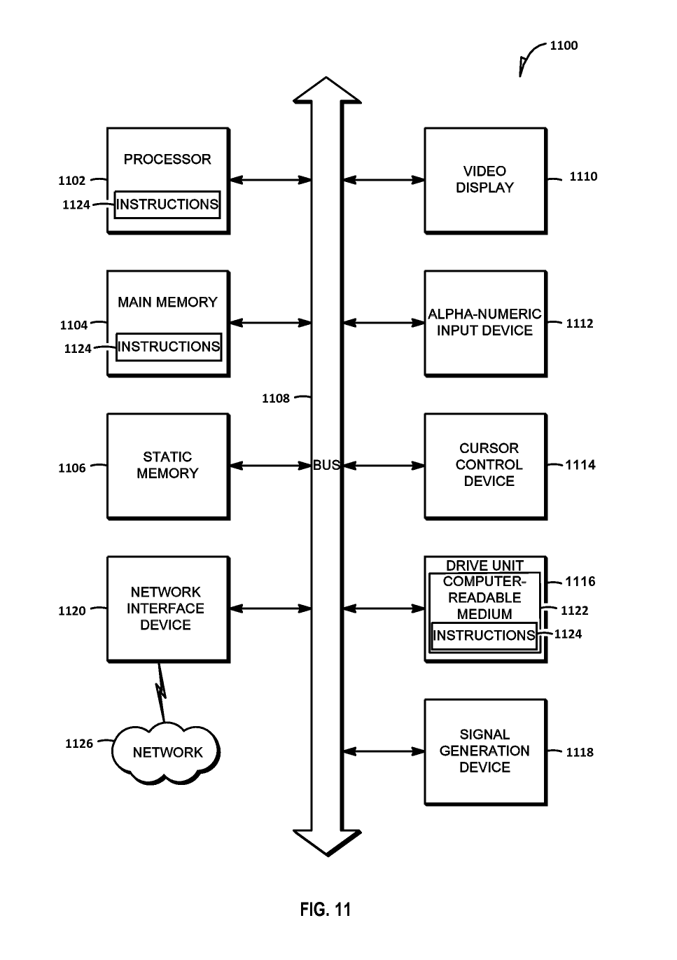

FIG. 11 is a block diagram of a machine in the example form of a computer system 1100 within which instructions for causing the machine to perform any one or more of the methodologies discussed herein may be executed. In alternative embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1100 includes a processor 1102 (e.g., a central processing unit (CPU), a graphics processing unit (GPU) or both), a main memory 1104 and a static memory 1106, which communicate with each other via a bus 1108. The computer system 1100 may further include a video display unit 1110 (e.g., a liquid crystal display (LCD) or a cathode ray tube (CRT)). The computer system 1100 also includes an alphanumeric input device 1112 (e.g., a keyboard), a user interface (UI) navigation (or cursor control) device 1114 (e.g., a mouse), a disk drive unit 1116, a signal generation device 1118 (e.g., a speaker) and a network interface device 1120.

### Machine-Readable Medium

The disk drive unit 1116 includes a machine-readable medium 1122 on which is stored one or more sets of data structures and instructions 1124 (e.g., software) embodying or utilized by any one or more of the methodologies or functions described herein. The instructions 1124 may also reside, completely or at least partially, within the main memory 1104 and/or within the processor 1102 during execution thereof by the computer system 1100, the main memory 1104 and the processor 1102 also constituting machine-readable media. The instructions 1124 may also reside, completely or at least partially, within the static memory 1106.

While the machine-readable medium 1122 is shown in an example embodiment to be a single medium, the term “machine-readable medium” may include a single medium or multiple media (e.g., a centralized or distributed database, and/or associated caches and servers) that store the one or more instructions 1124 or data structures. The term “machine-readable medium” shall also be taken to include any tangible medium that is capable of storing, encoding or carrying instructions for execution by the machine and that cause the machine to perform any one or more of the methodologies of the present embodiments, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and compact disc-read-only memory (CD-ROM) and digital versatile disc (or digital video disc) read-only memory (DVD-ROM) disks.

### Transmission Medium

The instructions 1124 may further be transmitted or received over a communications network 1126 using a transmission medium. The instructions 1124 may be transmitted using the network interface device 1120 and any one of a number of well-known transfer protocols (e.g., HTTP). Examples of communication networks include a LAN, a WAN, the Internet, mobile telephone networks, POTS networks, and wireless data networks (e.g., WiFi and WiMax networks). The term “transmission medium” shall be taken to include any intangible medium capable of storing, encoding, or carrying instructions for execution by the machine, and includes digital or analog communications signals or other intangible media to facilitate communication of such software.

Although an embodiment has been described with reference to specific example embodiments, it will be evident that various modifications and changes may be made to these embodiments without departing from the broader spirit and scope of the present disclosure. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A device comprising:
at least one electrode configured to be attached to a user and to detect brainwave activity from the user; and
a hardware processor comprising an electroencephalogram (EEG) application configured to measure a voltage of the electrode, to compute a derivative of the voltage of the electrode, to compute a coefficient of variation from the derivative of the voltage, and to determine a quality of contact of the electrode to the user based on the coefficient of variation.

2. The device of claim 1, wherein the hardware processor is configured to determine a maximum coefficient of variation and a minimum coefficient of variation for the electrode.

3. The device of claim 2, wherein the hardware processor is configured to determine that the coefficient of variation is between the minimum coefficient of variation and the maximum coefficient of variation, and to identify the voltage as valid and a contact of the electrode as good quality in response to determining that the coefficient of variation is between the minimum coefficient of variation and the maximum coefficient of variation.

4. The device of claim 2, wherein the hardware processor is configured to determine that the coefficient of variation is between the minimum coefficient of variation and the maximum coefficient of variation, and to identify the voltage as not valid and a contact of the electrode as bad quality in response to determining that the coefficient of variation outside a range between the minimum coefficient of variation and the maximum coefficient of variation.

5. The device of claim 1, wherein the hardware processor is configured to compute the standard deviation of the derivative of the voltage of the electrode, to compute the mean of the derivative of the voltage of the electrode, and to compute the coefficient of variation of the electrode by dividing the standard deviation of the derivative of the voltage of the electrode by the mean of the derivative of the voltage of the electrode.

6. The device of claim 1, wherein the quality of contact includes a Boolean value, the Boolean value including a good quality and a bad quality.

7. The device of claim 6, wherein the hardware processor is configured to determine a minimum false time duration as a time for a good contact quality of the electrode before identifying a bad contact quality of the electrode.

8. The device of claim 7, wherein the hardware processor is configured to identify the bad contact quality of the electrode in response to identifying bad contact quality for a period of time exceeding a predefined number of voltage computation cycles.

9. The device of claim 1, further comprising:
a camera configured to capture a reference identifier from a physical object,
wherein the processor further comprises an augmented reality application configured to identify a virtual object associated with the reference identifier, to display the virtual object in a display of the device, in response to a relative movement between the device and the physical object caused by a user, to modify the virtual object based on the quality of contact of the electrode to the user.

10. The device of claim 1, wherein the EEG application is configured to identify a change in a state of mind of the user of the device, wherein the augmented reality application is configured to modify the virtual object based on the change in the state of mind of the user of the device.

11. A method comprising:
measuring a voltage of an electrode attached to a user, the electrode configured to detect brainwave activity of the user;
computing a derivative of the voltage of the electrode, using a hardware processor;
computing a coefficient of variation from the derivative of the voltage; and
determining a quality of contact of the electrode to the user based on the coefficient of variation.

12. The method of claim 11, further comprising:
determining a minimum coefficient of variation and a maximum coefficient of variation for the electrode.

13. The method of claim 12, further comprising:
determining that the coefficient of variation is between the minimum coefficient of variation and the maximum coefficient of variation; and
identifying the voltage as valid and a contact of the electrode as good quality in response to determining that the coefficient of variation is between the minimum coefficient of variation and the maximum coefficient of variation.

14. The method of claim 12, further comprising:
determining that the coefficient of variation is between the minimum coefficient of variation and the maximum coefficient of variation; and
identifying the voltage as not valid and a contact of the electrode as bad quality in response to determining that the coefficient of variation outside a range between the minimum coefficient of variation and the maximum coefficient of variation.

15. The method of claim 11, further comprising:
computing the standard deviation of the derivative of the voltage of the electrode;
computing the mean of the derivative of the voltage of the electrode; and
computing the coefficient of variation of the electrode by dividing the standard deviation of the derivative of the voltage of the electrode by the mean of the derivative of the voltage of the electrode

16. The method of claim 11, wherein the quality of contact includes a Boolean value, the Boolean value including a good quality and a bad quality.

17. The method of claim 16, further comprising:
determining a minimum false time duration as a time for a good contact quality of the electrode before identifying a bad contact quality of the electrode.

18. The method of claim 17, further comprising:
identifying the bad contact quality of the electrode in response to identifying bad contact quality for a period of time exceeding a predefined number of voltage computation cycles.

19. The device of claim 11, further comprising:
a camera configured to capture a reference identifier from a physical object,
wherein the processor further comprises an augmented reality application configured to identify a virtual object associated with the reference identifier, to display the virtual object in a display of the device, in response to a relative movement between the device and the physical object caused by a user, to modify the virtual object based on the quality of contact of the electrode to the user.

20. A non-transitory machine-readable medium comprising instructions that, when executed by one or more processors of a machine, cause the machine to perform operations comprising:
detecting brainwave activity from the user with at least one electrode connected to a user; and
computing a voltage of the electrode using a hardware processor;
computing a derivative of the voltage of the electrode;
computing a coefficient of variation from the derivative of the voltage; and
determining a quality of contact of the electrode to the user based on the coefficient of variation.
